(19) United States
(12) Patent Application Publication
(10) Pub. No.: US 2020/0157531 A1
(43) Pub. Date: May 21, 2020

(54) METHOD TO ENHANCE SCREENING FOR HOMOLOGOUS RECOMBINATION IN GENOME EDITED CELLS USING RECOMBINATION-ACTIVATED FLUORESCENT DONOR DELIVERY VECTOR

(71) Applicant: Wisconsin Alumni Research Foundation, Madison, WI (US)

(72) Inventors: David V. Thompson, Monona, WI (US); Mark E. Burkard, Cross Plains, WI (US)

(21) Appl. No.: 16/684,191

(22) Filed: Nov. 14, 2019

Related U.S. Application Data
(60) Provisional application No. 62/768,676, filed on Nov. 16, 2018.

(51) Int. Cl. C12N 15/10; C12N 15/85; C12N 9/22; C12N 15/11

(52) U.S. Cl. CPC C12N 15/1082; C12N 15/85; C12N 2310/20; C12N 15/11; C12N 2800/80; C12N 9/22

(57) ABSTRACT

Constructs, vectors, and methods for enhancing homology directed repair using the CRISPR/Cas9 gene editing platform are disclosed.

### CROSS-REFERENCE TO RELATED APPLICATIONS

This application claims priority to U.S. Provisional Application No. 62/768,676, filed Nov. 16, 2018, which is incorporated by reference herein and relied on in its entirety.

### REFERENCE TO A SEQUENCE LISTING SUBMITTED VIA EFS-WEB

The content of the ASCII text file of the sequence listing named “960296_03964_ST25.txt” which is 12.2 kb in size was created on Nov. 14, 2019 and electronically submitted via EFS-Web herewith the application is incorporated herein by reference in its entirety.

### BACKGROUND

The CRISPR/Cas9 nuclease system has emerged as a promising new option for genome editing. Using CRISPR/Cas9 to create indels (deletions or insertions) in a genome is relatively easy. However, performing gene editing is often difficult and inefficient. The insertion of precise genetic modifications by genome editing tools such as CRISPR/Cas9 is limited by the relatively low efficiency of homology-directed repair (HDR) compared with the higher efficiency of the nonhomologous end-joining (NHEJ) pathway. This is a widely accepted problem, and many have attempted to address this through a host of different approaches for promoting HDR (often at the expense of NHEJ). Nonetheless, this remains a real limitation on the efficient use of CRISPR for gene editing.

A need in the art exists for improved methods of CRISPR/Cas9 HDR for genome editing technologies.

### SUMMARY OF THE INVENTION

In a first aspect, described herein is a nucleic acid construct comprising a gene that encodes for a first selectable marker comprising a 5′ portion that comprises a direct repeat at the 3′ end of said 5′ portion; and a 3′ portion that comprises the direct repeat at the 5′ end of said 3′ portion, wherein the 5′ portion and the 3′ portion are separated by a multiple cloning site. In some embodiments, the selectable marker is a fluorescent protein. In some embodiments, the construct is a vector.

In some embodiments, the fluorescent protein is selected from the group consisting of a green fluorescent protein, a red fluorescent protein, a blue fluorescent protein, a cyan fluorescent protein, a yellow fluorescent protein, an orange fluorescent protein, and a far-red fluorescent protein

In some embodiments, the selectable marker is an antibiotic resistance marker.

In some embodiments, the multiple cloning site is a restriction enzyme cleavage site selected from the group consisting of Nhe1, EcoRV, Sac1, AflII, AlfI, ArsI, AscI, AsiSI, BaeI, BarI, BbvCI, BclI, BmgBI, Bpu10I, BsiWI, BsmBI, BspEI, BsrGI, BstBI, BstB17I, ClaI, CspCI, DraIII, EcoNI, EcoRI, FseI, HpaI, MauBI, MfeI, MluI, NruI, NsiI, PacI, PasI, PmeI, PmlI, PpuMI, PshAI, PsrI, RsrII, SanDI, SgrDI, SphI, SrfI, SwaI, TstI, Tth111I, XcmI, and Xho1 cleavage sites.

In a second aspect, described herein is a nucleic acid construct comprising a gene that encodes for a selectable marker comprising a 5′ portion that comprises a direct repeat at the 3′ end of said 5′ portion; and a 3′ portion that comprises the direct repeat at the 5′ end of said 3′ portion, wherein the 5′ portion and the 3′ portion are separated by a donor repair template. In some embodiments, the donor repair template is between about 400 base pairs and about 1000 base pairs.

In some embodiments, the selectable marker is a fluorescent protein. In some embodiments, the fluorescent protein is selected from the group consisting of a green fluorescent protein, a red fluorescent protein, a blue fluorescent protein, a cyan fluorescent protein, a yellow fluorescent protein, an orange fluorescent protein, and a far-red fluorescent protein.

In some embodiments, the selectable marker is an antibiotic resistance marker selected from the group consisting of an ampicillin resistance marker, a kanamycin resistance marker, a chloramphenicol resistance marker, a puromycin resistance marker, a hygromycin resistance marker, a blasticidin resistance marker, a neomycin resistance marker, and a zeocin resistance marker. In some embodiments, the selectable marker is a β-galactosidase or a luciferase selectable marker.

In a third aspect, described herein is a system for CRISPR/Cas9 gene editing comprising a nucleic acid donor repair construct as described herein and including a first selectable marker and a construct encoding a Cas9 nuclease, a guide RNA (gRNA), and a second selectable marker. In some embodiments, the first and second selectable markers are fluorescent proteins.

In a fourth aspect, described herein is a method of screening for homology directed repair (HDR) comprising the steps of: transfecting a population of cells with a first construct comprising a gene that encodes a first selectable marker comprising: a 5′ portion that comprises a direct repeat at the 3′ end of said 5′ portion; and a 3′ portion that comprises the direct repeat at the 5′ end of said 3′ portion, wherein the 5′ portion and the 3′ portion are separated by a donor repair template; transfecting the population of cells with a second construct comprising a gene encoding a Cas9 nuclease, a sequence encoding a guide RNA, and a gene that encodes a second selectable marker; and selecting cells from the population that are positive for expression of both the first and second selectable marker, whereby the selected cells are enriched for HDR.

In some embodiments, the first and second selectable markers are fluorescent proteins of different wavelengths. In some embodiments, the fluorescent proteins are selected from the group consisting of a green fluorescent protein, a red fluorescent protein, a blue fluorescent protein, a cyan fluorescent protein, a yellow fluorescent protein, an orange fluorescent protein, a far-red fluorescent protein.

In some embodiments, the first construct and the second construct are included on the same plasmid for transfection into the population of cells. In some embodiments, the first construct and the second construct are on separate plasmids for transfection into the population of cells.

In some embodiments, the cells are transfected using lipid based transfection, nucleofection, or viral transfection. In some embodiments, the population of cells are plant cells, animal cells, eukaryotic cells, prokaryotic cells, mammalian cells, bacterial cells, fungal cells, nematode cells, or insect cells.

In some embodiments, the cells are selected using fluorescence-activated cells sorting (FACS). In some embodiments, the cells are selected using antibiotic resistance selection. In some embodiments, the antibiotic resistance marker is selected from the group consisting of an ampicillin resistance marker, a kanamycin resistance marker, a chloramphenicol resistance marker, a puromycin resistance marker, a hygromycin resistance marker, a blasticidin resistance marker, a neomycin resistance marker, and a zeocin resistance marker.

In some embodiments, the cells are selected using bioluminescence screening or β-galactosidase screening.

### BRIEF DESCRIPTION OF DRAWINGS

The patent or patent application file contains at least one drawing in color. Copies of this patent or patent application publication with color drawings will be provided by the Office upon request and payment of the necessary fee.

FIG. 1 shows CRISPR/Cas9 mediated genome editing. A guide RNA (indicated as sgRNA in FIG. 1) binds the Cas9 nuclease and guides the nuclease to the target sequence of the gene of interest, which is upstream of a protospacer adjacent motif (PAM). The Cas9 nuclease and the sgRNA may be transfected into a cells and expressed from a plasmid or may be introduced as a purified Cas9 protein and a synthetic sgRNA. Once bound at the target site, the Cas9 nuclease induces a double strand break at the target site. If no donor sequence is inserted, non-homologous end joining (NHEJ) will result in an insertion or deletion (indel) mutation which may result in a frame shift mutation or other disruption in transcription and expression of the gene of interest. If a donor sequence is present, homology directed repair (HDR) may result in the desired precise gene editing at the target site, but this occurs at low efficiency as NHEJ also can occur.

FIG. 2 shows CRISPR editing workflow.

FIG. 3 shows schematics for the surveyor assay and the restriction digest assay used in traditional CRISPR genome editing screening.

FIG. 4 shows plasmids used in CRISPR editing for selection of cells positive for expression of the Cas9 nuclease by GFP or puromycin selection. Plasmids encode both the gRNA and the Cas9 nuclease as well as a selectable marker. In the embodiments depicted the selectable marker is either a fluorescent marker (GFP) or an antibiotic selectable marker (puromycin). Expression of both the Cas9 nuclease and the selectable marker are controlled by the same promoter (for example, a CMV promoter) and selection for cells positive for expression of the selectable marker will be positive for Cas9 nuclease expression.

FIG. 5 shows the design of a plasmid vector 100 for delivery of repair donors into the genome via HDR (Homology Directed Repair) with CRISPR Cas9 editing protocols. The vector 100 contains an RFP gene (or a gene encoding any other fluorescent protein or a gene encoding an antibiotic resistance marker) interrupted by two identical 223 bp direct repeats 106. These 223 bp repeats code for a segment of the RFP gene. In the vector the RFP gene is broken up into a 5′ portion 102 and a 3′ portion 104. The 3′ end of the 5′ portion 102 is the first 223 bp direct repeat 106. The 5′ end of the 3′ portion 104 is the second 223 bp direct repeat 106. Between the two 223 bp direct repeats 106 is the repair donor template sequence 108. The circular (non-linearized) vector with the repeats will not express RFP when transfected into cells. Nor does the circular repair vector express RFP when a repair donor is cloned between the direct repeats. After transfection into cells, a fraction of the repair donor fragments will recombine by HDR into the genome at a specific location determined by the Cas9/sgRNA and this will linearize the repair RFP vector. The linearized repair donor will recombine at the direct repeats and produce functional RFP gene expression.

FIGS. 6A-6C show the method for enhanced screening for CRISPR HDR edits using the repair donor vector of the present invention. FIG. 6A shows a method for enhanced screening for HDR genome editing of cells using a fluorescent vector (RFP) with direct repeats 106 to deliver the HDR repair donor 108. A second vector contains the Cas9 protein, gRNA sequence and GFP. It is also envisioned that all components may be encoded on a single vector. FIG. 6B shows cleavage of the genomic DNA by Cas9 allows site specific HDR recombination of the repair donor 108 into the genome. FIG. 6C shows recombination of the repair donor vector at the direct repeats 106 producing RFP fluorescence.

FIGS. 7A-7E show additional embodiments of the donor repair vector of the present invention. FIG. 7A shows the same embodiment depicted in FIG. 5 including the 5′ portion 102 of the RFP gene with the first direct repeat 106 and the 3′ portion 104 of the RFP gene with the second direct repeat 106 interrupted by the repair donor sequence 108. FIG. 7B shows an additional circular embodiment including the 5′ portion 102 of the RFP gene with the first direct repeat 106 and the 3′ portion 104 of the RFP gene with the second direct repeat 106 interrupted by the repair donor sequence 108 and also including one Cas9 wild type (WT) guide cleavage site 110. FIG. 7C shows an additional circular embodiment including the 5′ portion 102 of the RFP gene with the first direct repeat 106 and the 3′ portion 104 of the RFP gene with the second direct repeat 106 interrupted by the repair donor sequence 108 and also including two Cas9 WT guide cleavage sites 110. FIG. 7D shows an additional circular embodiment including the 5′ portion 102 of the RFP gene with the first direct repeat 106 and the 3′ portion 104 of the RFP gene with the second direct repeat 106 interrupted by a Cas9 WT guide cleavage site 110. In this embodiment, a single stranded oligodeoxynucleotide (ODN) repair oligo is co-transfected with the reporter vector and a vector expressing the Cas9 nuclease. In some embodiments, the ODN is about 100 bp and contains the mutation to be introduced into the genome. The ODN serves as the repair donor and cells that are positive for Cas9 cleavage will cleave the WT cleavage site 110 in the RFP repair vector to produce RFP fluorescence. FIG. 7E shows a linear plasmid embodiment including the 5′ portion 102 of the RFP gene with the first direct repeat 106 and the 3′ portion 104 of the RFP gene with the second direct repeat 106 interrupted by the repair donor sequence 108 and additional including a restriction enzyme cleavage site 112.

FIG. 8 shows enhanced selection for HDR using the repair donor template of the present invention with a Cas9 encoding vector including a selectable marker. In this embodiment, the selectable marker on the Cas9 encoding vector is GFP and the selectable marker on the repair donor vector is RFP, therefore FACs sorting of GFP/RFP positive cells will select for cells in which the desired HDR event was successful.

FIG. 9 shows that the linearized donor repair delivery vector recombines and expresses RFP after homologous recombination in mammalian HEK cells.

FIG. 10 shows HEK cells co-transfected with Cas9-GFP vector and linearized donor repair RFP vector. Green cells indicate cells position for Cas9 plasmid only (Cas9/GFP expression). Red cells indicate cells positive for the linearized donor repair RFP vector expressing RFP after recombination. Orange and yellow cells indicate cells positive for the Cas9 plasmid and the donor repair RFP vector. These cells contain both the Cas9 vector required for genomic DNA cleavage and recombined donor repair vector.

FIG. 11 shows results using two different embodiments of the present invention for enhanced HDR screening. Two different forms of the repair donor delivery plasmid (linearized and circular) were transfected in cells along with the Cas9/sgRNA. After 72 hours cells were FACs sorted for GFP only cells (standard screening method) and GFP+RFP expressing cells (enhanced screening method disclosed herein). Using both linear and circular forms of the repair donor delivery plasmid showed a significant difference in cells containing the Cas9 guide only (GFP sort) vs. cells containing Cas9 guide and repair donor plasmid (GFP/RFP sort).

FIG. 12 shows screening of GFP and GFP/RFP FACs sorted pools by restriction enzyme digest. Sorted cell pools 1 and 2 show the results of HEK cell transfection using linearized donor repair plasmid along with GFP expressing Cas9/gRNA vectors. Sorted cell pools 3 and 4 show the results of HEK cell transfections using circular donor repair plasmid along with GFP expressing Cas9/gRNA vectors. Cell pools 1 and 3 were FACs sorted for GFP expressing cells only (standard screen). Cell pools 2 and 4 were FACs sorted for both RFP and GFP (enhanced screen disclosed herein). Based on restriction enzyme digest assays to screen for positive HDR, only 21% and 19.5% of cells transfected with linearized or circular plasmids, respectively, were positive when selection was based on GFP expression only. When cells were selected based on GFP and RFP expression using the donor repair vector constructions of the present invention, cells positive for HDR increased to 31.2% and 38.8% for cells transfected with linearized and circular plasmids, respectively.

FIG. 13 shows results in HEK cells co-transfected with Cas9-GFP plasmid and the donor repair vector (RFP). Cell pools 1 and 2 were screened by FACs sorting for cells expressing GFP only (standard screen) and cells were then plated to obtain individual clones. Cell pools 3 and 4 were FACs sorted for cells expressing both GFP and RFP (enhanced screen disclosed herein) and cells were then plated to obtain individual clones. These clones were then analyzed by PCR amplification across the region of interest and by restriction enzyme digest with Tsp451 indicated donor insertion. For pools 1 (linear donor plasmid) and 2 (circular donor plasmid) 66% and 16% of cells, respectively, were positive for HDR. When cells were selected for using both GFP and RFP using the donor repair template constructs of the present invention, the cells positive for HDR increased to 92% and 75% for groups 3 (linear) and 4 (circular), respectively. PCR products indicated by the red starts were selected for sequencing to confirm donor template insertion.

FIG. 14 show sequencing results of the PCR products from GFP sorted clones in FIG. 13. Results indicate that cells positive for Tsp451 digest have been edited with the repair donor. For linear GFP sort pool 1, clones A and B are positive for Tsp451 digest indicating donor insertion. Sequence data confirms mixture of donor and WT sequences indicating a heterologous edit (one allele). For circular GFP sort pool 2, clones A and B are negative for Tsp451 digest indicating no donor insertion. Sequence data shows clone A has a deletion indicating NHEJ editing. Clone B shows WT sequence indicating no editing occurred.

FIG. 15 shows sequencing results of the PCR products from the GFP/RFP pools in FIG. 13. For linear GFP/RFP sort pool 3, clones A and B are positive for Tsp451 digest indicating donor insertion. Sequence data confirms a mixture of donor and WT sequences indicating a heterozygous edit (editing on one allele). For circular GFP/RFP sorted pool 4, clone A is negative for Tsp451 digest indicated no donor insertion. Clone A was sequenced as a negative control. Sequence data show clone A has a deletion indicating NHEJ editing. The sequence data for clone B confirm a mixture of donor and WT sequences indicating a heterozygous edit (editing on one allele).

FIG. 16 shows an example of a modified donor delivery plasmid as described herein. The backbone of the plasmid is Addgene plasmid #11151. The DsRed2 gene is modified to include a 230 bp direct repeat with cloning sites in between the repeats.

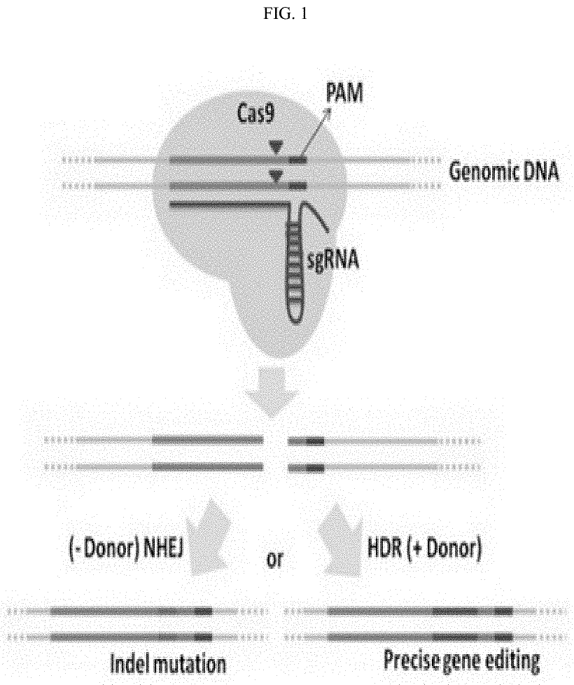

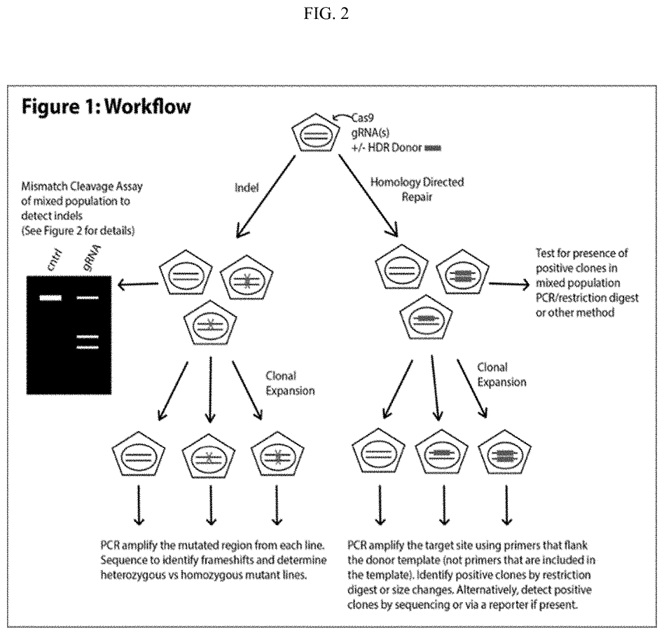

### INCORPORATION BY REFERENCE

All publications, including but not limited to patents and patent applications, cited in this specification are herein incorporated by reference as though set forth in their entirety in the present application.

### DETAILED DESCRIPTION OF THE INVENTION

CRISPR/Cas9 works by inducing sequence-specific double-stranded breaks (DSBs) in DNA. After such breaks, the cell undergoes an error-prone repair process called non-homologous end joining (NHEJ), leading to a disruption in the translational reading frame, often resulting in frameshift mutations and premature stop codons. Alternatively, if a repair template is provided, the cell may undergo homology directed repair (HDR) to incorporate the sequence of the repair template into the site of the double-strand break.

For the system to work for HDR, at least three components must be introduced in cells: a Cas9 nuclease, a guide RNA, and a repair template. For standard CRISPR methods known in the art, the repair template may be provided as part of a double stranded donor plasmid, as a double stranded PCR product, or as a single stranded oligonucleotide. The repair template includes the desired mutation to be introduced at the Cas9 cleavage site or any desired sequence to be incorporated into the genome at the Cas9 cleavage site.

Described herein are repair template constructs and methods of use for enhanced HDR directed genome editing.

The present disclosure describes vectors and methods for enhanced screening for homologous recombination genome editing of cells using a vector including a selectable marker interrupted with direct repeats to deliver a repair template. The vector is constructed such that a selectable marker will only be expressed upon removal of the repair template from the vector and recombination of the direct repeats to form the complete and uninterrupted selectable marker gene from which the selectable marker is expressed.

### Constructs of the Present Invention

The present invention provides a donor repair template construct for transfection into a cell. The donor repair template construct includes a gene encoding a selectable marker. In the construct, the gene encoding the selectable marker is interrupted by two identical direct repeat sequences to form a 5′ portion of the selectable marker gene and a 3′ portion of the selectable marker gene. The direct repeat sequence is repeated first at the far 3′ end of the 5′ portion and again at the far 5′ end of the 3′ portion. In some embodiments, between the two identical direct repeat sequences is a donor repair template sequence. The construct is arranged such that upon removal of the repair template sequence, the direct repeat sequences recombine to form the full gene encoding the selectable marker and the selectable marker may be expressed. If the repair template sequence is not removed, the selectable marker cannot be expressed.

As used herein “repair template sequence” or “donor repair sequence” refers to the nucleotide sequence to be inserted at the site of Cas9 cleavage in the cell. The repair template sequence may be any nucleotide sequence and may be used to introduce mutations, including silent mutations, frameshift mutations, single nucleotide substitutions, and the like at the Cas9 cleavage site. The repair donor template sequence is about 400 base pairs (bp) to about 1000 bp. The repair donor template sequence may be about 400, about 500, about 600, about 700, about 800, about 900, or about 1000 base pairs. The length of the repair donor template will depend on the nature of the mutation to be made and the cloning method used to insert the donor repair template sequence into the construct.

In some embodiments, between the two identical direct repeat sequences is a multiple cloning site for cloning of a donor repair template sequence into the vector. As used herein “multiple cloning site” refers to a nucleotide sequence used for controlled cloning of a desired sequence into a construct at a predetermined location. The multiple cloning site may be any sequence known in the art used for cloning. Multiple cloning sites for use in the donor repair template constructs of the present invention may include, but are not limited to, restriction enzyme cleavage sites. Suitable restriction enzyme cleavage sites will be cleavage sites that are not found anywhere else in the vector and are unique to the donor repair template construct. Restriction enzyme cleavage site include, but are not limited to, Nhe1, EcoRV, Sac1, AflII, AlfI, ArsI, AscI, AsiSI, BaeI, BarI, BbvCI, BclI, BmgBI, Bpu10I, BsiWI, BsmBI, BspEI, BsrGI, BstBI, BstB17I, ClaI, CspCI, DraIII, EcoNI, EcoRI, FseI, HpaI, MauBI, MfeI, MluI, NruI, NsiI, PacI, PasI, PmeI, PmlI, PpuMI, PshAI, PsrI, RsrII, SanDI, SgrDI, SphI, SrfI, SwaI, TstI, Tth111I, XcmI, and Xho1 cleavage sites. The DNA sequences associated with the restriction cleavage sites listed, as well as other known restriction enzyme cleavage sites, are known in the art.

In some embodiments, the repair donor and direct repeats are inserted without restriction enzymes as a double-stranded DNA or using alternative cloning methods such as Gibson assembly. The double-stranded DNA may be produced synthetically (e.g., chemical synthesis or gBlock™) or by polymerase chain reaction and used to assemble the donor repair template construct. When Gibson assembly methods are used the donor repair template may be up to about 500 bp.

As used herein “selectable marker” refers to a protein or nucleic acid used to sort or select a given population of cells based on a characteristic property of said maker. The selectable marker is encoded by a gene for said selectable marker. Suitable selectable markers include, but are not limited to, fluorescent proteins, antibiotic resistance markers, a β-galactosidase selectable marker, and a luciferase selectable marker. The selectable marker gene may be a gene encoding a fluorescent protein, a gene encoding an antibiotic resistance marker, a gene encoding a β-galactosidase, or a gene encoding a luciferase. Selectable markers, and methods of selecting cells using said selectable markers are known in the art. See, for example, Mortensen et al. (“Selection of transfected mammalian cells,” Current Protocols in Neuroscience, 1997) and Patrick (“Plasmids 101: Mammalian Vectors,” Addgene Blog, 2014).

In some embodiments, the selectable marker is a fluorescent protein. Fluorescent proteins may include, but are not limited to, a green fluorescent protein (e.g., GFP), a red fluorescent protein (e.g., RFP), a blue fluorescent protein, a cyan fluorescent protein, a yellow fluorescent protein, an orange fluorescent protein, a far-red fluorescent protein, and the like. Fluorescent proteins and gene sequences encoding fluorescent proteins are known in the art (Kremers et al. “Fluorescent proteins at a glance,” Journal of Cell Science, 124, 157-160, 2011).

In some embodiments, the selectable marker is an antibiotic resistance marker which, when expressed, confers antibiotic resistance to the cell. Antibiotic resistance markers may include, but are not limited to, ampicillin resistance markers, kanamycin resistance markers, chloramphenicol resistance markers, puromycin resistance markers, hygromycin resistance markers, blasticidin resistance markers, neomycin (G418/Geneticin) resistance markers, zeocin resistance markers, and the like.

In some embodiments, the selectable marker is a β-galactosidase selectable marker. β-galactosidase selectable markers and methods of selecting cells using β-galactosidase selectable markers are known in the art. See, for example, Weir et al. (“The use of beta-galactosidase as a marker gene to define the regulatory sequences of the herpes simplex virus type 1 glycoprotein C gene in recombinant herpesviruses,” Nuc. Acids Res., 1988, 16 (21): 10267-10282).

In some embodiments, the selectable marker is a luciferase selectable marker and the cells are selected based on bioluminescence screening. Luciferase selectable markers and methods of selecting cells using bioluminescence are known in the art. See, for example, Brasier et al. (“Optimized use of the firefly luciferase assay as a reporter gene in mammalian cell lines,” BioTechniques, 1989 7 (10):1116-1122).

As used herein “direct repeat sequence” refers to a portion of the selectable marker gene which is repeated twice in the vector between the 5′ and 3′ ends of the selectable marker gene. The direct repeat may be between about 10 bp to about 250 bp long. In some embodiments, the direct repeat may be at least about 10, about 20, about 30, about 40, about 50, about 60, about 70, about 80, about 90, about 100, about 110, about 120, about 130, about 140, about 150, about 160, about 180, about 200, about 225, or about 250 base pairs (bp) long. The upper limit on the length of the direct repeat will vary depending on the length of the gene encoding the selectable marker. For example, the RFP gene is approximately 700 bp so the upper limit of the direct repeat when the selectable marker is RFP will be less than 700 bp. Without wishing to be bound by any particular theory, the more homology, that is the longer the direct repeat, the more likely it will be that recombination will occur and the smaller the direct repeats, the less efficient recombination will be. In general, a direct repeat of at least 10 bp will be sufficient for homologous recombination to occur. It is known in the art that changing the length of the direct repeat will alter the efficiency of homologous recombination, and a skilled artisan will understand what minimum and maximum lengths are suitable for the direct repeats in various constructs comprising various selectable markers and donor repair template sequences. See, for example, Rubnitz et al. (“The minimum amount of homology required for homologous recombination in mammalian cells,” Molecular and Cellular Biology, 1984, 4 (11):2253-2258), Perez et al. (“Factors affecting double-stranded break-induced homologous recombination in mammalian cells,” BioTechniques, 2005, 39:109-115), Zhang et al. (“Efficient precise knocking with a double cut HDR donor after CRISPR/Cas9-mediated double-strand DNA cleavage,” Genome Biology, 2017, 18:35), and Fujimoto et al. (“Minimum length of homology arms required for effective red/ET recombination,” Biosci. Biotechnol. Biochem., 73 (12), 2783-2786, 2009).

When the selectable marker is interrupted in the vector, the direct repeat region will be repeated at both the 3′ most end of the 5′ portion of the selectable marker gene and the 5′ most end of the 3′ portion of the selectable marker gene. For example, in embodiments where the direct repeat is 100 bp, the last 100 bp of the 5′ portion of the selectable marker gene will be defined as the direct repeat and will match the first 100 bp of the 3′ portion of the selectable marker gene. In another example, when the direct repeat is 80 bp, the last 80 bp of the 5′ portion of the selectable marker gene will be defined as the direct repeat and will match the first 80 bp of the 3′ portion of the selectable maker gene.

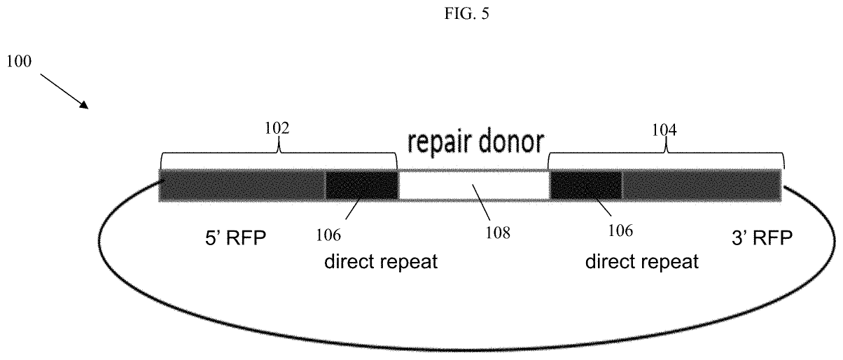

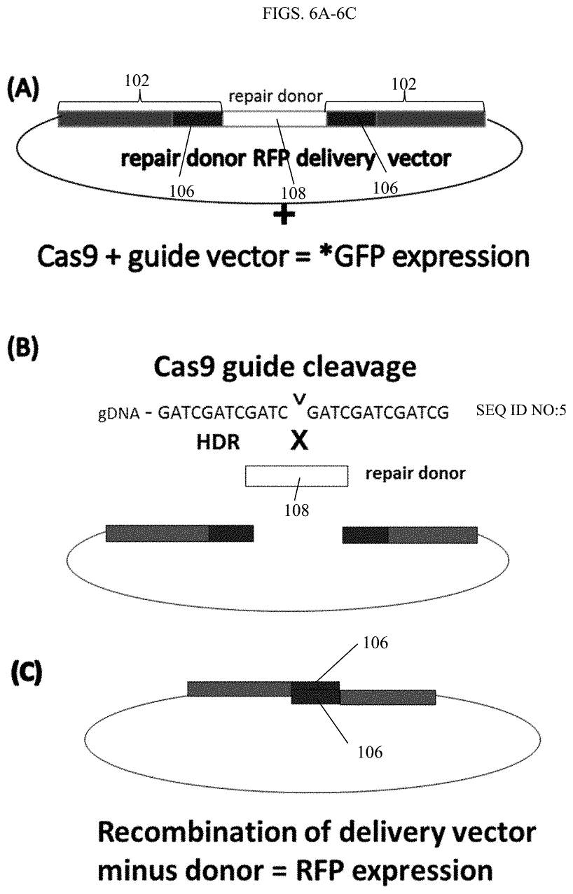

FIG. 5 shows one embodiment of a donor repair template construct in a vector. In this embodiment, the vector 100 contains a red fluorescent protein (RFP) reporter gene interrupted by two identical 223 bp direct repeats 106 such that a 5′ portion 102 of the RFP gene and a 3′ portion 104 of the RFP are present in the construct. The first 223 bp direct repeat 106 is at the far 3′ end of the 5′ portion 102 and the second 223 bp direct repeat 106 is at the far 5′ end of the 3′ portion 104. In this embodiment, the direct repeats 106 are separated by a repair donor template sequence 108. While this embodiment shows the use of the RFP reporter gene, it is understood that other selectable marker genes may be used and assembled in the same construct.

In addition to the repair donor construct, constructs of the present invention may include a nucleotide encoding a Cas9 nuclease and a guide RNA (gRNA). The one or more constructs encoding the Cas9 nuclease and the gRNA will also include a selectable marker. In some embodiments the sequence encoding the Cas9 nuclease and the gRNA are included on a single vector construct. In some embodiments, the repair donor construct, the sequence encoding the Cas9 nuclease and the gRNA are included in a single vector. In some embodiments, the repair donor construct is on a vector separate from the construct for the Cas9 nuclease and the gRNA. Additionally, the Cas9 and gRNA constructs may include a promoter, a poly(A) tail, and an optional reporter element. In some embodiments, the Cas9 nuclease may be provided to the cell as a purified protein and the gRNA and tracrRNA may be provided as a separate synthesized or transcribed RNA.

As used herein “guide RNA (gRNA)” refers to the nucleotide guide sequence which directs Cas9 mediated cleavage at a target site specific and complementary to the target region of the gRNA. The gRNA may be between about 15 to about 20 bp (e.g., 15 bp, 16 bp, 17 bp, 18 bp, 19 bp, or 20 bp). The gRNA may be specific to any target site suitable for Cas9 mediated cleavage. The gRNA is fused to or hybridized to the tracrRNA sequence for binding to the Cas9 nuclease. While the target region of the gRNA sequence is variable and will be specific for the cleavage site of interest, the tracrRNA is the same for all gRNA sequences used and specific for binding to the Cas9 nuclease. The tracrRNA sequence for S. pyrogenes Cas9 is GTTTTAGAGCTAGAAATAGCAAGTTAAAATAAGGCTAGTCCGTTATCAACTTGAAAA AGTGGCACCGAGTCGGTGC (SEQ ID NO:1).

The donor repair template construct may additionally include one or more cleavage sites. In some embodiments, the cleavage site is identical to the target site of the gRNA used for CRISPR/Cas9 mediated HDR. The cleavage sites may be between the donor repair fragment and the direct repeat at either the 5′ or 3′ end of the donor repair fragment. Cleavage at these sites within the donor repair template construct are a method to linearize the donor repair vector and increase the efficiency of recombination.

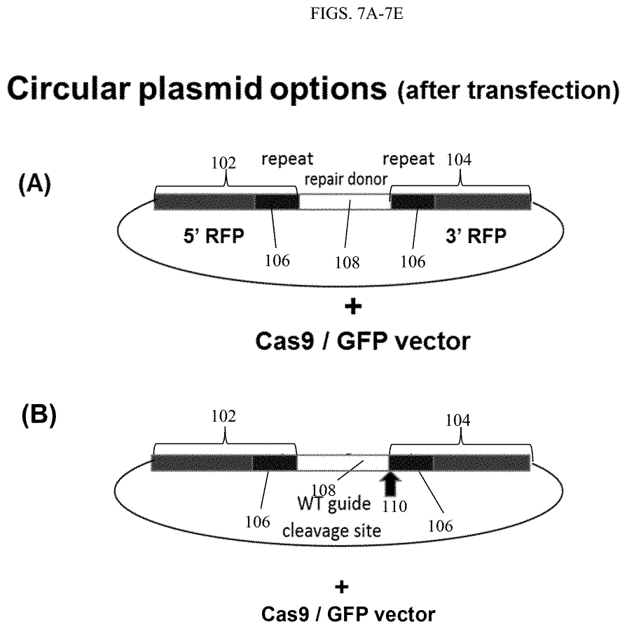

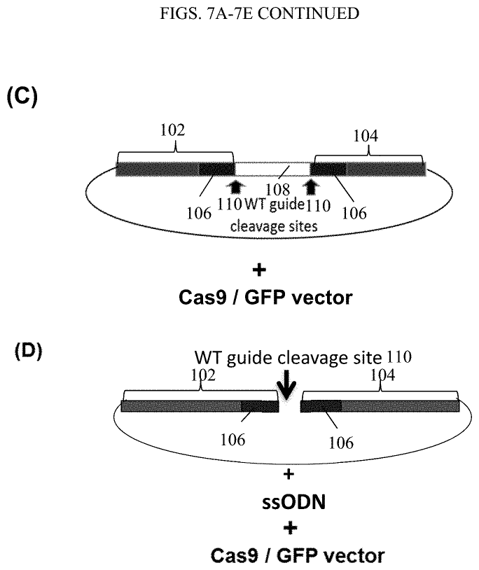

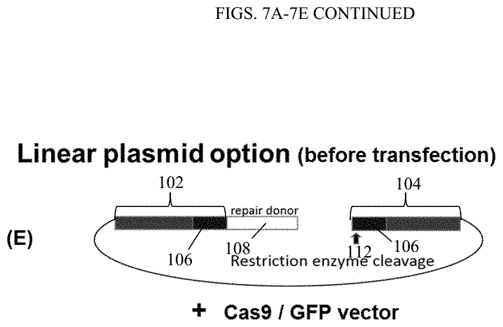

FIG. 7B shows one embodiment of a donor repair template construct in a vector. In this embodiment, the vector contains a red fluorescent protein (RFP) reporter gene interrupted by two identical 223 bp direct repeats 106 such that a 5′ portion 102 of the RFP gene and a 3′ portion 104 of the RFP are present in the construct. The first 223 bp direct repeat 106 is at the far 3′ end of the 5′ portion 102 and the second 223 bp direct repeat 106 is at the far 5′ end of the 3′ portion 104. In this embodiment, the direct repeats 106 are separated by repair donor template sequence 108 and a gRNA target site 110 is included before the second direct repeat.

FIG. 7C shows one embodiment of a donor repair template construct in a vector. In this embodiment, the vector contains a red fluorescent protein (RFP) reporter gene interrupted by two identical 223 bp direct repeats 106 such that a 5′ portion 102 of the RFP gene and a 3′ portion 104 of the RFP are present in the construct. The first 223 bp direct repeat 106 is at the far 3′ end of the 5′ portion 102 and the second 223 bp direct repeat 106 is at the far 5′ end of the 3′ portion 104. In this embodiment, the direct repeats 106 are separated by repair donor template sequence 108. This embodiment also includes two gRNA target sites 110 included between each direct repeat and the donor repair template. The gRNA target sites do not need to be directly between the direct repeat and the donor repair template and there is flexibility in the placement of the target sites.

FIG. 7D shows one embodiment of a donor repair template construct in a vector. In this embodiment, the vector contains a red fluorescent protein (RFP) reporter gene interrupted by two identical 223 bp direct repeats 106 such that a 5′ portion 102 of the RFP gene and a 3′ portion 104 of the RFP are present in the construct. The first 223 bp direct repeat 106 is at the far 3′ end of the 5′ portion 102 and the second 223 bp direct repeat 106 is at the far 5′ end of the 3′ portion 104. In this embodiment, the direct repeats 106 are separated by a gRNA target site 110. For methods using this construct, the donor repair template is introduced into the cell as a separate single stranded DNA oligonucleotide.

The donor repair template construct may additionally include one or more restriction enzyme cleavage sites. Restriction enzyme cleavage sites for use in the present invention include, but are not limited to, cleavage sites for Nhe1, EcoRV, Sac1, AflII, AlfI, ArsI, AscI, AsiSI, BaeI, BarI, BbvCI, BclI, BmgBI, Bpu10I, BsiWI, BsmBI, BspEI, BsrGI, BstBI, BstB17I, ClaI, CspCI, DraIII, EcoNI, EcoRI, FseI, HpaI, MauBI, MfeI, MluI, NruI, NsiI, PacI, PasI, PmeI, PmlI, PpuMI, PshAI, PsrI, RsrII, SanDI, SgrDI, SphI, SrfI, SwaI, TstI, Tth111I, XcmI, and Xho1.

FIG. 7E shows one embodiment of a linearized donor repair template construct in a vector. In this embodiment, the vector contains a red fluorescent protein (RFP) reporter gene interrupted by two identical 223 bp direct repeats 106 such that a 5′ portion 102 of the RFP gene and a 3′ portion 104 of the RFP are present in the construct. The first 223 bp direct repeat 106 is at the far 3′ end of the 5′ portion 102 and the second 223 bp direct repeat 106 is at the far 5′ end of the 3′ portion 104. In this embodiment, the direct repeats 106 are separated by repair donor template sequence 108. This embodiment also includes a restriction enzyme cleavage site 112. Cleavage of the vector using the restriction enzyme specific for the restriction enzyme cleavage site 112 will generate a linearized plasmid. The restriction enzyme cleavage site may be between the first direct repeat 106 and the 5′ end of the repair donor template sequence or between the 3′ end of the repair donor template sequence and the second direct repeat 106.

Constructs may be packaged in a vector suitable for delivery into a cell, including but not limited to an adeno-associated viral (AAV) vector, a lentiviral vector, a retroviral vector or a vector suitable for transient transfection. Suitable vector backbones are known and commercially available in the art. In some embodiments, the vector is an AAV vector and the donor repair, gRNA, and Cas9 constructs are encoded on separate vectors. In some embodiments, the vector is an AAV vector and the donor repair, gRNA, and Cas9 constructs are encoded on a single vector. In some embodiments, the vector is a vector suitable for transient transfection and the donor repair, gRNA, and Cas9 constructs are encoded on a single vector. In some embodiments, the vector is a lentiviral vector and the donor repair, gRNA, and Cas9 constructs are encoded on separate vectors. In some embodiments, the vector is a lentiviral vector and the donor repair, gRNA, and Cas9 constructs are encoded on a single vector. In some embodiments, the vector is a vector suitable for transient transfection and the donor repair, gRNA, and Cas9 constructs are encoded on a single vector. Suitable CRISPR viral vectors are known and used in the art.

Constructs of the present invention may be transfected into a cell using any suitable transfection reagent or transfection method known in the art. Construction may be transfected using lipid transfection (e.g., lipofectamine), electroporation (e.g., Thermo Neon™ electroporation), nucleofection (e.g., Lonza Nucleofector™), viral transfection, and calcium phosphate transfection. Suitable transfection systems and methods are known in the art.

### Methods of the Present Invention

Provided herein is a method for screening a population of cells for HDR. The population of cells is transfected with a donor repair template construct. The donor repair template construct for transfection may be any donor repair construct described herein and includes a first selectable marker and a repair template sequence. The population of cells are also transfected with a construct expressing a Cas9 nuclease, a gRNA, and a second selectable marker. The gRNA may be introduced to the cells by expression from a vector or as a synthetic oligonucleotide. In some embodiments, the donor repair construct and the Cas9 nuclease/gRNA construct are transfected into the population of cells on separate vectors. In some embodiments, the donor repair construct and the Cas9 nuclease/gRNA construct are transfected into the population of cells as a single vector.

Cells to be transfected with the constructs of the present invention can be any suitable cell type capable of being transformed by methods known in the art. Suitable cells may include, but are not limited to, plant cells, animal cells, eukaryotic cells, prokaryotic cells, mammalian cells, bacterial cells, fungal cells, nematode cells, or insect cells.

For methods of enhanced HDR screening described herein, the first selectable marker in the donor repair construct will be different than the second selectable marker in the Cas9 nuclease construct. For example, if the first selectable maker in the donor repair construct is RFP, the second selectable marker in the Cas9 nuclease construct will not be RFP but rather will be a different selectable maker, such as GFP, another fluorescent protein, or an antibiotic resistance marker. The first and second selectable markers may be any selectable maker so long as they are different selectable makers. In some embodiments, the first selectable marker and the second selectable marker are fluorescent proteins. In some embodiments, the first selectable marker and the second selectable marker are an antibiotic resistance marker. In some embodiments, the first selectable marker is a fluorescent protein and the second selectable marker is an antibiotic resistance marker. In some embodiment, the first selectable marker is an antibiotic resistance marker and the second selectable marker is a fluorescent protein.

Following transfection of the cells with the donor repair construct and the Cas9 nuclease/gRNA construct, the population of cells are sorted based expression of both the first and second selectable markers. The cells may be sorted by any means known in art for selecting for expression of the selectable markers. A skilled artisan understands the appropriate methods to use for selection of cells expressing a given selectable markers.

For embodiments in which the selectable marker is a fluorescent protein, the cells may be sorted using fluorescent-activated cell sorting (FACS) and flow cytometry. In FACS sorting, cells are sorted based on the specific light scattering and fluorescent characteristics of each cell. When multiple fluorescent proteins are used to select for cells comprising multiple constructs, it is advantageous to choose fluorescent proteins with distinct excitation and emission peaks to be targeted. A skilled artisan will understand how to select suitable fluorescent markers or other selectable markers to ensure suitable sorting and identification of cells.

In some embodiments, cell or colonies of cells may be sorted using microscopy techniques. Visualization and mechanical sorting of cells may be used when the selectable markers used impart visual differences in the cells positive for Cas9 nuclease/gRNA or donor template construct expression.

For embodiments in which the selectable marker is an antibiotic resistance marker, the cells may be sorted by growing or culturing the cells in the presence of the antibiotic corresponding to the antibiotic resistance marker used and the cell line being transfected. For example, in embodiments where the antibiotic resistance marker is a puromycin resistance gene, growth or culture of the cells in the presence of puromycin will select for the cells expressing the puromycin resistance gene. A skilled artisan will understand suitable selection methods as taught in the art.

The selected population of cells will be enriched for cells expressing the Cas9 nuclease/gRNA and cells in which the HDR event was successful determined by removal of the donor repair template from the donor repair construct and recombination of the selectable marker in the donor repair vector.

In some embodiments, following enrichment of the population of cells by selection using the first and second selectable markers, the cells are further screened using a surveyor assay or a restriction digest assay.

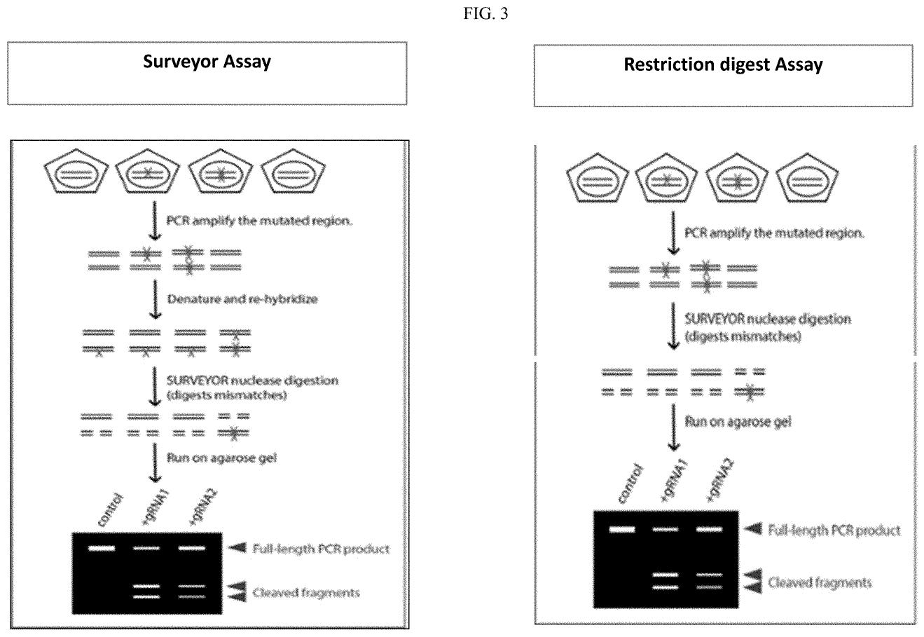

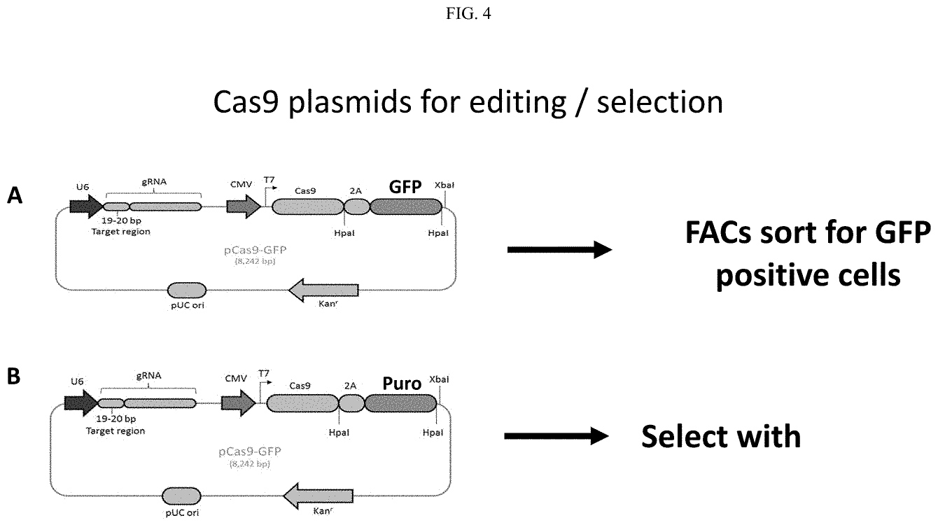

In some embodiments, methods of screening for HDR or NHEJ include a surveyor assay. FIG. 3 shows an example of the surveyor assay. The surveyor assay is a screening method for checking cell pools after CRISPR editing and selection. The sequence of the target region of the genome corresponding to the gRNA target site is known, and PCR primers are designed to amplify the target region of the genome. In some embodiments, the PCR primers are designed to amplify an approximately 500 bp fragment of the target region. Genomic DNA is extracted from cells in the pools of interest. The genomic DNA then serves as the templates for PCR amplification using the designed PCR primers. Generally, the genomic DNA will include a mixture of edited and wild type of unedited cells. Therefor the PCR amplification product will also include a mixture of edited and WT DNA. The amplified fragment is then denatured and rehybridized to form PCR products that contain (i) double stranded WT (unedited) DNA; (ii) double stranded, edited, DNA; and (iii) heterozygous double-stranded DNA wherein one strand, a first strand, is WT DNA and the other strand, a second strand, is edited DNA. The heterozygous double-stranded DNA will not hybridize completely due to base pair mismatches. Mismatched base pair are recognized using the Surveyor nuclease enzyme which cleaves the mismatches. All rehybridized DNA is exposed to the Surveyor nuclease enzyme. DNA exposed to the Surveyor enzyme is run on an agarose gel and if cleavage products are present, the cell pool necessarily includes at least some cells that have been edited.

In some embodiments, methods for screening for HDR include a restriction digest assay. FIG. 3 shows an example of the restriction digest assay. The restriction digest assay is a screening method for checking cell pools after CRISPR editing and selection. The restriction digest assay is useful if a new restriction site is introduced in the donor sequence that is recombined into the genome by HDR. The sequence of the target region of the genome corresponding to the gRNA target site is known, and PCR primers are designed to amplify the target region of the genome. In some embodiments, the PCR primers are designed to amplify an approximately 500 bp fragment of the target region. Genomic DNA is extracted from cells in the pools of interest. The genomic DNA then serves as the templates for PCR amplification using the designed PCR primers. Generally, the genomic DNA will include a mixture of edited and wild type of unedited cells. Therefor the PCR amplification product will also include a mixture of edited and WT DNA. Following amplification, the PCR amplification product is exposed to a restriction digest enzyme. The restriction enzyme used will be specific for the restriction enzyme cleavage site introduced in the donor repair sequence. The digested DNA is run on an agarose gel and if cleavage products are present, the donor sequence has been inserted into the genomic DNA. This is a positive indication that some cells in the screened pool are positive for HDR editing.

The present invention has been described in terms of one or more preferred embodiments, and it should be appreciated that many equivalents, alternatives, variations, and modifications, aside from those expressly stated, are possible and within the scope of the invention.

### Example 1

The embodiment described here demonstrates enhanced screening and enrichment for HDR in genome edited cells using GFP and RFP selectable markers.

FIG. 5 shows the design of a plasmid vector for delivery of repair donors into the genome via HDR (Homology Directed Repair) with CRISPR Cas9 editing protocols. The vector contains an RFP gene interrupted by two identical 223 bp direct repeats. The circular vector with the repeats will not express RFP when transfected into cells. Nor does the circular repair vector express RFP when a repair donor is cloned between the direct repeats. RFP will be expressed when the donor repair template is removed during successful HDR.

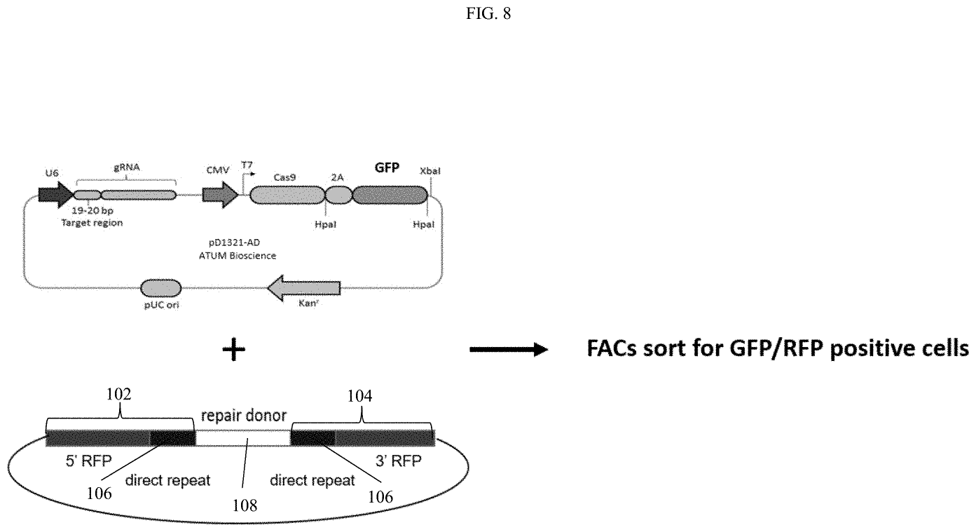

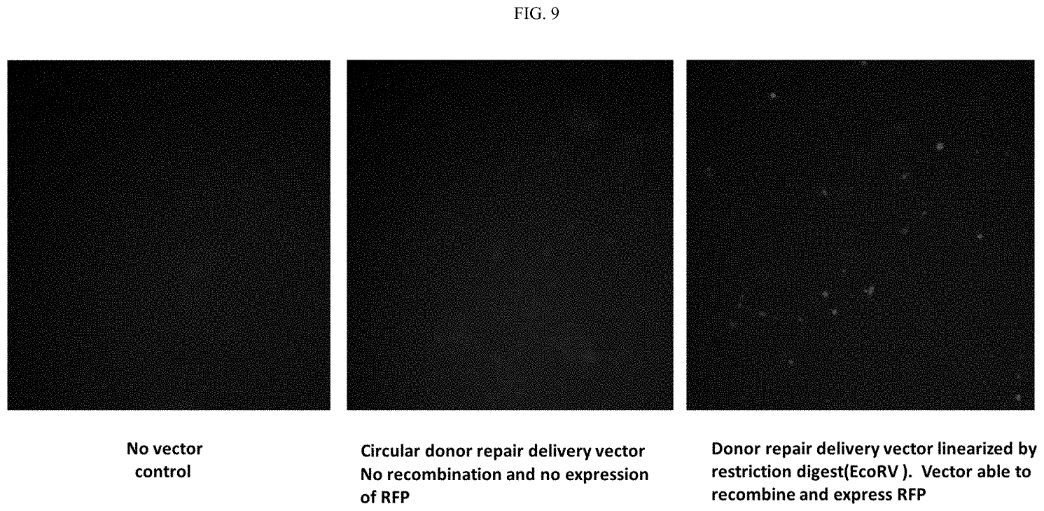

FIG. 8 shows both the repair donor vector and the Cas9/gRNA vector used in these experiments. The Cas9/gRNA vector includes a GFP selectable marker which is expressed under the same promoter as the Cas9 nuclease. Therefore, cells positive for GFP expression will indicate cells in which the Cas9 nuclease is also expressed.

Human embryonic kidney (HEK) cells (ATCC® CRL-1573) were transfected with the RFP donor repair vector and the GFP Cas9/nuclease vectors described in FIG. 8 using the lipid transfection reagent lipofectamine using lipid transfection methods.

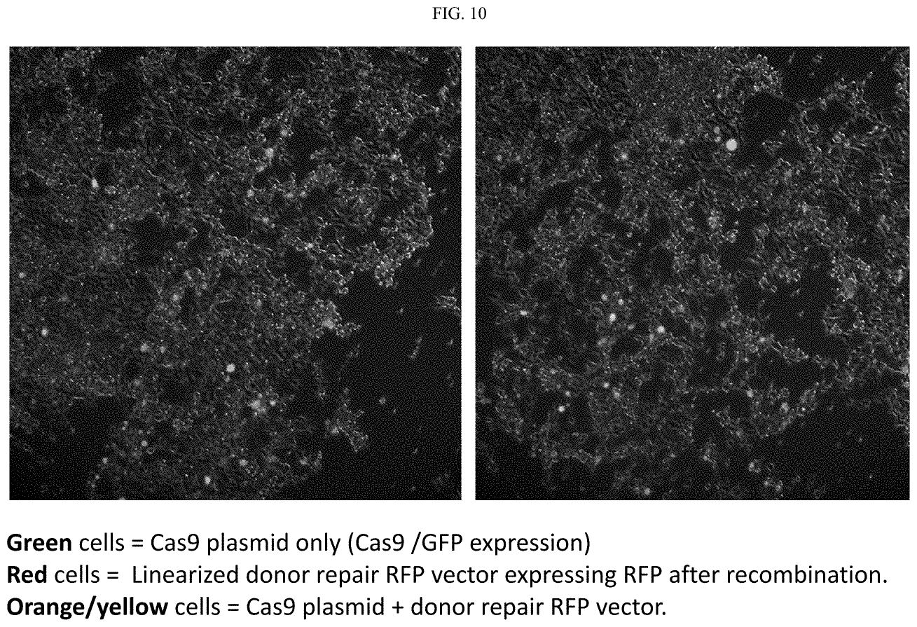

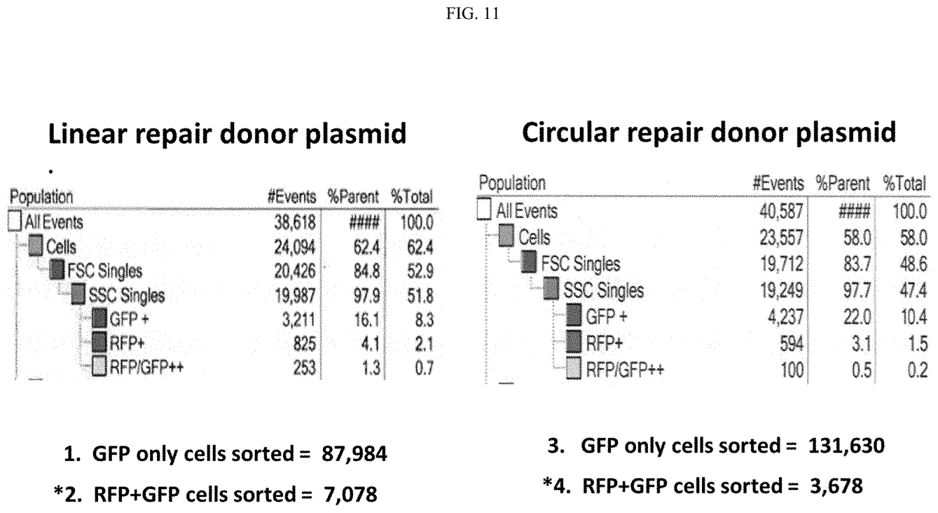

After transfection into cells, a fraction of the repair donor fragments will recombine by HDR into the genome at a specific location determined by the Cas9/sgRNA and this will linearize the repair RFP vector. The linearized repair donor will recombine and produce functional RFP gene expression. As depicted in FIG. 10, cells positive for only the Cas9/gRNA plasmid are green, cells expressing only the linearized RFP donor vector are red and cells that include both the Cas9/gRNA vector and the donor vector will appear orange or yellow.

FIG. 11 shows the results using two different donor repair vector constructs for enhanced HDR screening. Linearized and circular donor repair vectors with an RFP selectable marker were transfected along with the Cas9/gRNA expression construct, which expresses GFP, into cells using lipofectamine and lipid transfection methods. After 72 hours cells were FACs sorted for GFP only cells (standard screening method) and GFP+RFP expressing cells. A summary of the cell pools are included below in Table 1. Using both linear and circular forms of the repair donor delivery plasmid showed a significant difference in cells containing the Cas9 guide only (GFP sort) vs. cells containing Cas9 guide and repair donor plasmid (GFP/RFP sort).

TABLE 1
Quantification of cell pools sorted
for GFP or GFP + RFP expression

| Donor Vector | FACs sort | Total Cells |
|---|---|---|
| Linear | GFP only | 87,984 |
| Linear | GFP/RFP | 7,078 |
| Circular | GFP only | 131,630 |
| Circular | GFP/RFP | 3,678 |

The GFP/RFP sorted cell pools have eliminated a large number of the unedited cells or false positives seen when cells were sorted for GFP expressing cells only. Cells only expressing GFP, and not expressing RFP, containing the Cas9/gRNA vector only. There is no indication if these cells contain the donor template or if HDR was successful. The GFP/RFP cells contain the Cas9/gRNA vector as well as the repair donor vector in which recombination and HDR have occurred. The repair donor vector has lost the donor fragment because it has recombined to create RFP expression. If the repair donor has been removed from the repair donor vector we assume it has been recombined into the genome by HDR. The difference in GFP only sorted cells compared to the GFP/RFP sorted cells is significant. The smaller the pool of sorted cells, the more efficient the screening for edited cells and the better the odds of finding HDR edited cells.

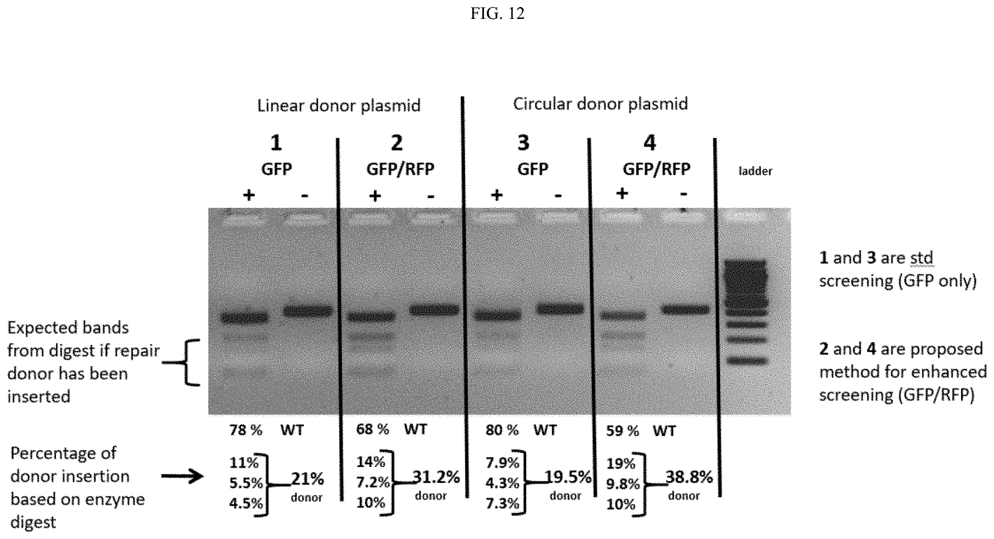

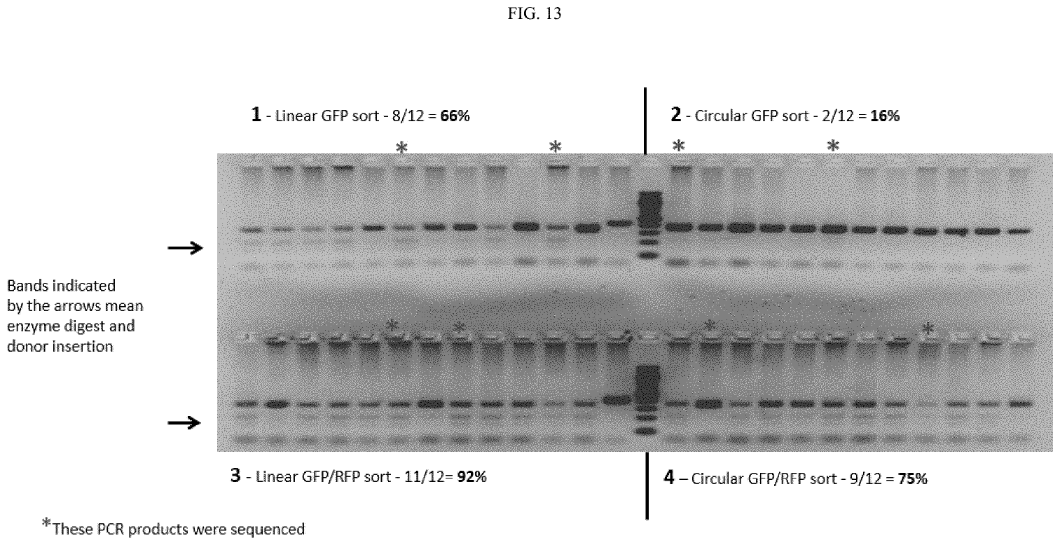

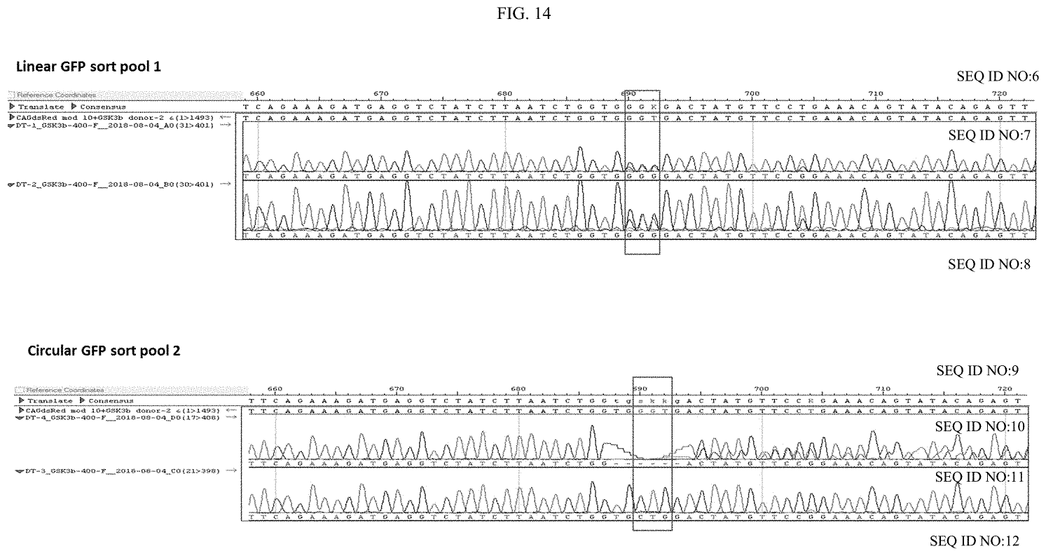

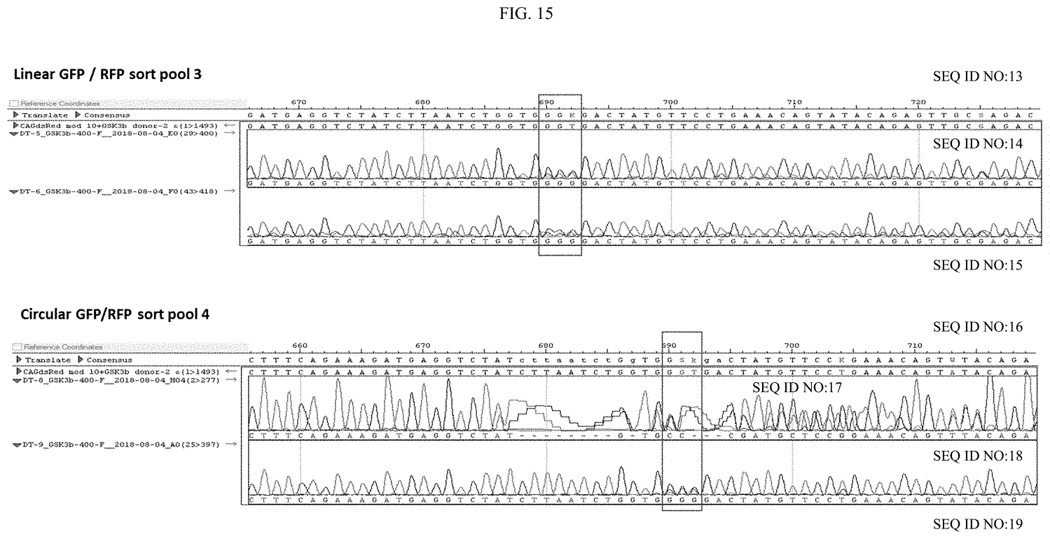

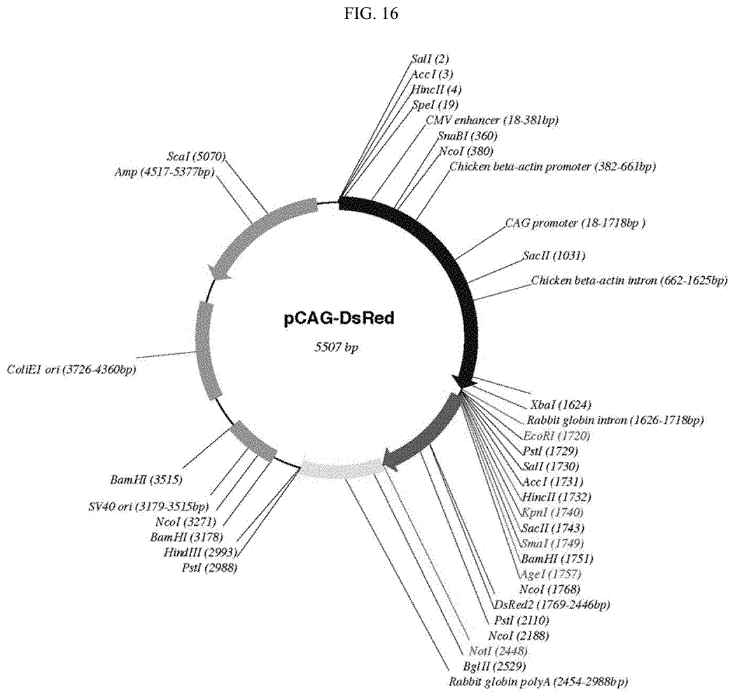

FIG. 12 shows the results of screening the GFR and GFP/RFP sorted pools using the restriction digest assay. FIGS. 13-15 show the results of the experiments in HEK cells and sequencing of genomic DNA extracted therefrom. Cells sorted for GFP/RFP expressed showed a higher rate of successful HDR events with the donor sequence of interest.

An example of the donor repair template construct is included below as SEQ ID NO:2. Direct repeats are in BOLD (SEQ ID NO:3) and the restriction digest sites separating the direct repeats are underlined (SEQ ID NO:4).

GTCGACATTGATTATTGACTAGTTATTAATAGTAATCAATTACGGGGTCATTAGTTC
ATAGCCCATATATGGAGTTCCGCGTTACATAACTTACGGTAAATGGCCCGCCTGGCT
GACCGCCCAACGACCCCCGCCCATTGACGTCAATAATGACGTATGTTCCCATAGTAA
CGCCAATAGGGACTTTCCATTGACGTCAATGGGTGGACTATTTACGGTAAACTGCCC
ACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTA
CTTGGCAGTACATCTACGTATTAGTCATCGCTATTACCATGGGTCGAGGTGAGCCCC
ACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGCGCGCGCCA
GGCGGGGCGGGGCGGGGCGAGGGGCGGGGCGGGGCGAGGCGGAGAGGTGCGGCG
GCAGCCAATCAGAGCGGCGCGCTCCGAAAGTTTCCTTTTATGGCGAGGCGGCGGCG
GCGGCGGCCCTATAAAAAGCGAAGCGCGCGGCGGGCGGGAGTCGCTGCGTTGCCTT
CGCCCCGTGCCCCGCTCCGCGCCGCCTCGCGCCGCCCGCCCCGGCTCTGACTGACCG
CGTTACTCCCACAGGTGAGCGGGCGGGACGGCCCTTCTCCTCCGGGCTGTAATTAGC
GCTTGGTTTAATGACGGCTCGTTTCTTTTCTGTGGCTGCGTGAAAGCCTTAAAGGGCT
CCGGGAGGGCCCTTTGTGCGGGGGGGAGCGGCTCGGGGGGTGCGTGCGTGTGTGTG
TGCGTGGGGAGCGCCGCGTGCGGCCCGCGCTGCCCGGCGGCTGTGAGCGCTGCGGG
CGCGGCGCGGGGCTTTGTGCGCTCCGCGTGTGCGCGAGGGGAGCGCGGCCGGGGGC
GGTGCCCCGCGGTGCGGGGGGGCTGCGAGGGGAACAAAGGCTGCGTGCGGGGTGTG
TGCGTGGGGGGGTGAGCAGGGGGTGTGGGCGCGGCGGTCGGGCTGTAACCCCCCCC
TGCACCCCCCTCCCCGAGTTGCTGAGCACGGCCCGGCTTCGGGTGCGGGGCTCCGTG
CGGGGCGTGGCGCGGGGCTCGCCGTGCCGGGCGGGGGGTGGCGGCAGGTGGGGGT
GCCGGGCGGGGCGGGGCCGCCTCGGGCCGGGGAGGGCTCGGGGGAGGGGCGCGGC
GGCCCCGGAGCGCCGGCGGCTGTCGAGGCGCGGCGAGCCGCAGCCATTGCCTTTTA
TGGTAATCGTGCGAGAGGGCGCAGGGACTTCCTTTGTCCCAAATCTGGCGGAGCCG
AAATCTGGGAGGCGCCGCCGCACCCCCTCTAGCGGGCGCGGGCGAAGCGGTGCGGC
GCCGGCAGGAAGGAAATGGGCGGGGAGGGCCTTCGTGCGTCGCCGCGCCGCCGTCC
CCTTCTCCATCTCCAGCCTCGGGGCTGCCGCAGGGGGACGGCTGCCTTCGGGGGGGA
CGGGGCAGGGCGGGGTTCGGCTTCTGGCGTGTGACCGGCGGCTCTAGAGCCTCTGCT
AACCATGTTCATGCCTTCTTCTTTTTCCTACAGCTCCTGGGCAACGTGCTGGTTATTG
TGCTGTCTCATCATTTTGGCAAAGAATTCTGCAGTCGACGGTACCGCGGGCCCGGGA
TCCACCGGTCGCCACCATGGCCTCCTCCGAGAACGTCATCACCGAGTTCATGCGCTT
CAAGGTGCGCATGGAGGGCACCGTGAACGGCCACGAGTTCGAGATCGAGGGCGAG
GGCGAGGGCCGCCCCTACGAGGGCCACAACACCGTGAAGCTGAAGGTGACCAAGG
GCGGCCCCCTGCCCTTCGCCTGGGACATCCTGTCCCCCCAGTTCCAGTACGGCTCCA
AGGTGTACGTGAAGCACCCCGCCGACATCCCCGACTACAAGAAGCTGTCCTTCC
CCGAGGGCTTCAAGTGGGAGCGCGTGATGAACTTCGAGGACGGCGGCGTGGCGACC
GTGACCCAGGACTCCTCCCTGCAGGACGGCTGCTTCATCTACAAGGTGAAGTTCA
TCGGCGTGAACTTCCCCTCCGACGGCCCCGTGATGCAGAAGAAGACCATGGGC
TGGGAGGCCTCCACCGAGCGCCTGTACCCCCGCGACGGCGTGCTGAAGGGCG
AGACCCACAAGGCCCTGAAGCTGAAGGACGGCGGCCACTACCTGGTGGAGTTC
AAGTCCATCTACATGGCCAAGAAGCCCGTGCAGGCTAGCGATATCGAGCTCCTCG
AGGGACGGCTGCTTCATCTACAAGGTGAAGTTCATCGGCGTGAACTTCCCCTC
CGACGGCCCCGTGATGCAGAAGAAGACCATGGGCTGGGAGGCCTCCACCGAG
CGCCTGTACCCCCGCGACGGCGTGCTGAAGGGCGAGACCCACAAGGCCCTGAA
GCTGAAGGACGGCGGCCACTACCTGGTGGAGTTCAAGTCCATCTACATGGCCA
AGAAGCCCGTGCAGCTGCCCGGCTACTACTACGTGGACGCCAAGCTGGACATCAC
CTCCCACAACGAGGACTACACCATCGTGGAGCAGTACGAGCGCACCGAGGGCCGCC
ACCACCTGTTCCTGTAGCGGCCGCACTCCTCAGGTGCAGGCTGCCTATCAGAAGGTG
GTGGCTGGTGTGGCCAATGCCCTGGCTCACAAATACCACTGAGATCTTTTTCCCTCT
GCCAAAAATTATGGGGACATCATGAAGCCCCTTGAGCATCTGACTTCTGGCTAATAA
AGGAAATTTATTTTCATTGCAATAGTGTGTTGGAATTTTTTGTGTCTCTCACTCGGAA
GGACATATGGGAGGGCAAATCATTTAAAACATCAGAATGAGTATTTGGTTTAGAGTT
TGGCAACATATGCCATATGCTGGCTGCCATGAACAAAGGTGGCTATAAAGAGGTCA
TCAGTATATGAAACAGCCCCCTGCTGTCCATTCCTTATTCCATAGAAAAGCCTTGAC
TTGAGGTTAGATTTTTTTTATATTTTGTTTTGTGTTATTTTTTTCTTTAACATCCCTAA
AATTTTCCTTACATGTTTTACTAGCCAGATTTTTCCTCCTCTCCTGACTACTCCCAGTC
ATAGCTGTCCCTCTTCTCTTATGAAGATCCCTCGACCTGCAGCCCAAGCTTGGCGTA
ATCATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGCCA
GCGGATCCGCATCTCAATTAGTCAGCAACCATAGTCCCGCCCCTAACTCCGCCCATC
CCGCCCCTAACTCCGCCCAGTTCCGCCCATTCTCCGCCCCATGGCTGACTAATTTTTT
TTATTTATGCAGAGGCCGAGGCCGCCTCGGCCTCTGAGCTATTCCAGAAGTAGTGAG
GAGGCTTTTTTGGAGGCCTAGGCTTTTGCAAAAAGCTAACTTGTTTATTGCAGCTTAT
AATGGTTACAAATAAAGCAATAGCATCACAAATTTCACAAATAAAGCATTTTTTTCA
CTGCATTCTAGTTGTGGTTTGTCCAAACTCATCAATGTATCTTATCATGTCTGGATCC
GCTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCGG
TATCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAAAATCGAC
GCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCAATGCTCACGCTGTAGGTATCT
CAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGG
ACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAG
CAGCAGATTACGCGCAGAAAAAAAGGATCTCAAGAAGATCCTTTGATCTTTTCTACG
GGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCA
ATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCG
TGTAGATAACTACGATACGGGAGGGCTTACCATCTGGCCCCAGTGCTGCAATGATAC
CGCGAGACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGTTGTT
GCCATTGCTACAGGCATCGTGGTGTCACGCTCGTCGTTTGGTATGGCTTCATTCAGCT
CCGGTTCCCAACGATCAAGGCGAGTTACATGATCCCCCATGTTGTGCAAAAAAGCG
GTTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATGCT
TTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTT
TAAAAGTGCTCATCATTGGAAAACGTTCTTCGGGGCGAAAACTCTCAAGGATCTTAC
CGCTGTTGAGATCCAGTTCGATGTAACCCACTCGTGCACCCAACTGATCTTCAGCAT
CTTTTACTTTCACCAGCGTTTCTGGGTGAGCAAAAACAGGAAGGCAAAATGCCGCAA
AAAAGGGAATAAGGGCGACACGGAAATGTTGAATACTCATACTCTTCCTTTTTCAAT
ATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTG

We claim:

1. A nucleic acid construct comprising:
a gene that encodes for a first selectable marker comprising:
a 5′ portion that comprises a direct repeat at the 3′ end of said 5′ portion; and
a 3′ portion that comprises the direct repeat at the 5′ end of said 3′ portion,
wherein the 5′ portion and the 3′ portion are separated by a multiple cloning site.

2. The nucleic acid construct of claim 1, wherein the selectable marker is a fluorescent protein.

3. The nucleic acid construct of claim 2, wherein the fluorescent protein is selected from the group consisting of a green fluorescent protein, a red fluorescent protein, a blue fluorescent protein, a cyan fluorescent protein, a yellow fluorescent protein, an orange fluorescent protein, a far-red fluorescent protein

4. The nucleic acid construct of claim 1, wherein the selectable marker is an antibiotic resistance marker.

5. The nucleic acid construct of claim 1, wherein the multiple cloning site is a restriction enzyme cleavage site selected from the group consisting of Nhe1, EcoRV, Sac1, AflII, AlfI, ArsI, AscI, AsiSI, BaeI, BarI, BbvCI, BclI, BmgBI, Bpu10I, BsiWI, BsmBI, BspEI, BsrGI, BstBI, BstB17I, ClaI, CspCI, DraIII, EcoNI, EcoRI, FseI, HpaI, MauBI, MfeI, MluI, NruI, NsiI, PacI, PasI, PmeI, PmlI, PpuMI, PshAI, PsrI, RsrII, SanDI, SgrDI, SphI, SrfI, SwaI, TstI, Tth111I, XcmI, and Xho1 cleavage sites.

6. The nucleic acid construct of claim 1, wherein the construct is a vector.

7. A nucleic acid construct comprising:
A gene that encodes for a selectable marker comprising:
a 5′ portion that comprises a direct repeat at the 3′ end of said 5′ portion; and
a 3′ portion that comprises the direct repeat at the 5′ end of said 3′ portion,
wherein the 5′ portion and the 3′ portion are separated by a donor repair template.

8. The nucleic acid construct of claim 7, wherein the selectable marker is a fluorescent protein.

9. The nucleic acid construct of claim 8, wherein the fluorescent protein is selected from the group consisting of a green fluorescent protein, a red fluorescent protein, a blue fluorescent protein, a cyan fluorescent protein, a yellow fluorescent protein, an orange fluorescent protein, a far-red fluorescent protein.

10. The nucleic acid construct of claim 7, wherein the selectable marker is an antibiotic resistance marker selected from the group consisting of an ampicillin resistance marker, a kanamycin resistance marker, a chloramphenicol resistance marker, a puromycin resistance marker, a hygromycin resistance marker, a blasticidin resistance marker, a neomycin resistance marker, and a zeocin resistance marker.

11. The nucleic acid construct of claim 7, wherein the donor repair template is between about 400 base pairs and about 1000 base pairs.

12. A system for CRISPR/Cas9 gene editing comprising:
the construct of claim 1; and
a construct encoding a Cas9 nuclease, a guide RNA (gRNA), and a second selectable marker.

13. The system of claim 12, wherein the first and second selectable markers are fluorescent proteins.

14. A method of screening for homology directed repair (HDR) comprising the steps of:
transfecting a population of cells with a first construct comprising a gene that encodes a first selectable marker comprising:
a 5′ portion that comprises a direct repeat at the 3′ end of said 5′ portion; and
a 3′ portion that comprises the direct repeat at the 5′ end of said 3′ portion,
wherein the 5′ portion and the 3′ portion are separated by a donor repair template;
transfecting the population of cells with a second construct comprising a gene encoding a Cas9 nuclease, a sequence encoding a guide RNA, and a gene that encodes a second selectable marker; and
selecting cells from the population that are positive for expression of both the first and second selectable marker, whereby the selected cells are enriched for HDR.

15. The methods of claim 14, wherein the first and second selectable markers are fluorescent proteins of different wavelengths.

16. The method of claim 15, wherein the fluorescent proteins are selected from the group consisting of a green fluorescent protein, a red fluorescent protein, a blue fluorescent protein, a cyan fluorescent protein, a yellow fluorescent protein, an orange fluorescent protein, a far-red fluorescent protein.

17. The method of claim 14, wherein the first construct and the second construct are included on the same plasmid for transfection into the population of cells.

18. The method of claim 14, wherein the first construct and the second construct are on separate plasmids for transfection into the population of cells.

19. The method of claim 14, wherein the cells are transfected using lipid based transfection, nucleofection, or viral transfection.

20. The method of claim 14, wherein the cells are selected using fluorescence-activated cells sorting (FACS).

21. The method of claim 14, wherein the cells are selected using antibiotic resistance selection.

22. The method of claim 21, wherein the antibiotic resistance marker is selected from the group consisting of an ampicillin resistance marker, a kanamycin resistance marker, a chloramphenicol resistance marker, a puromycin resistance marker, a hygromycin resistance marker, a blasticidin resistance marker, a neomycin resistance marker, and a zeocin resistance marker.

23. The method of claim 14, wherein the cells are selected using bioluminescence screening or β-galactosidase screening.

24. The nucleic acid construct of claim 1, wherein the selectable marker is a β-galactosidase or a luciferase selectable marker.

25. The nucleic acid construct of claim 7, wherein the selectable marker is a β-galactosidase or a luciferase selectable marker.


SEQUENCE LISTING (19 sequences)

SEQ ID NO: 1 - DNA, 76 bp, Streptococcus pyogenes
gttttagagctagaaatagcaagttaaaataaggctagtccgttatcaacttgaaaaagtggcaccgagtcggtgc

SEQ ID NO: 2 - DNA, 5754 bp, Artificial Sequence
  synthetic
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcaggctagcgatatcgagctcctcgagggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 3 - DNA, 223 bp, Artificial Sequence
  synthetic
ggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcag

SEQ ID NO: 4 - DNA, 24 bp, Artificial Sequence
  synthetic
gctagcgatatcgagctcctcgag

SEQ ID NO: 5 - DNA, 25 bp, Artificial Sequence
  synthetic
gatcgatcgatcgatcgatcgatcg

SEQ ID NO: 6 - DNA, 64 bp, Artificial Sequence
  synthetic
tcagaaagatgaggtctatcttaatctggtgggkgactatgttcctgaaacagtatacagagtt

SEQ ID NO: 7 - DNA, 64 bp, Artificial Sequence
  synthetic
tcagaaagatgaggtctatcttaatctggtgggtgactatgttcctgaaacagtatacagagtt

SEQ ID NO: 8 - DNA, 64 bp, Artificial Sequence
  synthetic
tcagaaagatgaggtctatcttaatctggtgggggactatgttccggaaacagtatacagagtt

SEQ ID NO: 9 - DNA, 64 bp, Artificial Sequence
  synthetic
ttcagaaagatgaggtctatcttaatctggtgskkgactatgttcckgaaacagtatacagagt

SEQ ID NO: 10 - DNA, 64 bp, Artificial Sequence
  synthetic
ttcagaaagatgaggtctatcttaatctggtgggtgactatgttcctgaaacagtatacagagt

SEQ ID NO: 11 - DNA, 58 bp, Artificial Sequence
  synthetic
ttcagaaagatgaggtctatcttaatctggactatgttccggaaacagtatacagagt

SEQ ID NO: 12 - DNA, 64 bp, Artificial Sequence
  synthetic
ttcagaaagatgaggtctatcttaatctggtgctggactatgttccggaaacagtatacagagt

SEQ ID NO: 13 - DNA, 64 bp, Artificial Sequence
  synthetic
gatgaggtctatcttaatctggtgggkgactatgttcctgaaacagtatacagagttgcsagac

SEQ ID NO: 14 - DNA, 64 bp, Artificial Sequence
  synthetic
gatgaggtctatcttaatctggtgggtgactatgttcctgaaacagtatacagagttgcgagac

SEQ ID NO: 15 - DNA, 64 bp, Artificial Sequence
  synthetic
gatgaggtctatcttaatctggtgggggactatgttcctgaaacagtatacagagttgcgagac

SEQ ID NO: 16 - DNA, 64 bp, Artificial Sequence
  synthetic
ctttcagaaagatgaggtctatcttaatctggtggskgactatgttcckgaaacagtwtacaga

SEQ ID NO: 17 - DNA, 64 bp, Artificial Sequence
  synthetic
ctttcagaaagatgaggtctatcttaatctggtgggtgactatgttcctgaaacagtatacaga

SEQ ID NO: 18 - DNA, 52 bp, Artificial Sequence
  synthetic
ctttcagaaagatgaggtctatgtgcccgatgctccggaaacagtttacaga

SEQ ID NO: 19 - DNA, 64 bp, Artificial Sequence
  synthetic
ctttcagaaagatgaggtctatcttaatctggtgggggactatgttcctgaaacagtatacaga